(19) United States
(12) Patent Application Publication
(10) Pub. No.: US 2025/0185919 A1
(43) Pub. Date: Jun. 12, 2025

(54) RELAXATION-BASED MAGNETIC RESONANCE THERMOMETRY WITH A LOW-FIELD SINGLE-SIDED MRI SCANNER

(71) Applicant: Promaxo, Inc., Oakland, CA (US)

(72) Inventors: Muller Francis De Matos Gomes, Alameda, CA (US); Srirama Venkataraman, Oakland, CA (US); Aleksandar Nacev, San Francisco, CA (US)

(21) Appl. No.: 18/762,386

(22) Filed: Jul. 2, 2024

Related U.S. Application Data
(63) Continuation of application No. PCT/US2022/082551, filed on Dec. 29, 2022.
(60) Provisional application No. 63/266,383, filed on Jan. 4, 2022.

(51) Int. Cl. A61B 5/01; A61B 5/055; G01R 33/48; G01R 33/56

(52) U.S. Cl. CPC A61B 5/015; A61B 5/055; G01R 33/4804; G01R 33/5602

(57) ABSTRACT

Disclosed is a magnetic imaging apparatus comprising a housing comprising a face, wherein a first axis extends through the face into a field of view, and an array of permanent magnets positioned in the housing, wherein an inherent gradient magnetic field extends from the array of permanent magnets relative to the first axis into the field of view. The magnetic imaging apparatus further comprises a gradient coil set, at least one radio frequency coil, a power circuit, a memory storing a relaxation model for a tissue type, and a control circuit. The control circuit is configured to obtain a T2 data set related to a structure positioned in the field of view, generate a T2-weighted image of the structure, and convert the T2-weighed image of the structure to a heat map based on the relaxation model for the tissue type.

### CROSS-REREFERENCE

This application is a continuation of International Patent Application No. PCT/US2022/082551, filed Dec. 12, 2022, which claims the benefit of U.S. Provisional Patent Application No. 63/266,383, filed on Jan. 4, 2022, which is hereby incorporated by reference in its entirety.

### BACKGROUND

Thermal ablations of soft tissues can be guided by measuring the temperature in vivo. However, it is difficult to measure temperature in vivo using existing systems in certain instances.

### SUMMARY

In one general aspect, the present disclosure provides a magnetic imaging apparatus comprising a housing comprising a face, wherein a first axis extends through the face into a field of view. The magnetic imaging apparatus further comprises an array of permanent magnets positioned in the housing, wherein an inherent gradient magnetic field extends from the array of permanent magnets relative to the first axis into the field of view. The magnetic imaging apparatus further comprises a gradient coil set, at least one radio frequency coil, a power circuit, a memory storing a relaxation model for a tissue type, and a control circuit in signal communication with the gradient coil set, the at least one radio frequency coil, the power circuit, and the memory. The control circuit is configured to obtain a T2 data set related to a structure positioned in the field of view, wherein the structure corresponds to the tissue type. The control circuit is further configured to generate a T2-weighted image of the structure, and convert the T2-weighed image of the structure to a heat map based on the relaxation model for the tissue type.

In another aspect, the present disclosure provides a single-sided magnetic imaging apparatus, comprising a housing comprising an array of permanent magnets, wherein an inherent gradient magnetic field extends from the array of permanent magnets relative to a first axis into a field of view, wherein the field of view is adjacent to the housing. The single-sided magnetic imaging apparatus further comprises a radio frequency coil, a power circuit coupled to the radio frequency coil, a memory storing relaxation models for tissue at different temperatures, and a control circuit. The control circuit is in signal communication with the radio frequency coil, the power circuit, and the memory. The control circuit is configured to transmit a waveform sequence to the radio frequency coil to produce an echo train sequence, obtain a T2 data set related to a structure positioned in the field of view, generate a T2-weighted image of the structure, and convert the T2-weighed image of the structure to a heat map based on the relaxation models stored in the memory.

In another aspect, the present disclosure provides a method of detecting temperature in an object of interest comprised of a first tissue type with a magnetic resonance imaging apparatus. The method comprises obtaining calibration data comprising T2 data for different temperatures for different tissue types including the first tissue type, generating models for the different tissue types from the calibration data, positioning the object of interest in a field of view, and transmitting a pulse sequence comprising sweeping frequency pulses. The method further comprises receiving T2 data corresponding to the object of interest, generating a T2-weighted image of the object of interest, and converting the T2-weighed image of the object of interest to a heat map based on the model for the first tissue type.

### BRIEF DESCRIPTION OF THE DRAWINGS

The novel features of the various aspects are set forth with particularity in the appended claims. The described aspects, however, both as to organization and methods of operation, may be best understood by reference to the following description, taken in conjunction with the accompanying drawings.

FIG. 1 is a perspective view of a magnetic resonance imaging (MRI) scanner, according to various aspects of the present disclosure.

FIG. 2 is an exploded, perspective view of the MRI scanner of FIG. 1, in which the permanent magnet assembly and the gradient coil sets within the housing are exposed, according to various aspects of the present disclosure.

FIG. 3 is an elevation view of the MRI scanner of FIG. 1, according to various aspects of the present disclosure.

FIG. 4 is an elevation view of the MRI scanner of FIG. 1, according to various aspects of the present disclosure.

FIG. 5 is a perspective view of the permanent magnet assembly of the MRI scanner of FIG. 1, according to various aspects of the present disclosure.

FIG. 6 is an elevation view of the gradient coil set and the permanent magnet assembly of the MRI system shown in FIG. 1, according to various aspects of the present disclosure.

FIG. 7 is a control schematic for a single-sided MRI system, according to various aspects of the present disclosure.

FIG. 8 is a schematic of the magnetic gradient along the Z axis, according to various aspects of the present disclosure.

FIG. 9 is a diagram of a pulse sequence that compensates for the varying field of view in slices along the Z axis, according to various aspects of the present disclosure.

FIG. 10 is a representative graph of a sweeping frequency pulse, according to various aspects of the present disclosure.

FIG. 11 is a diagram of a pulse sequence for collecting T2 relaxation times of a structure in the field of view, according to various aspects of the present disclosure.

FIG. 12 is a flow diagram for collecting calibration data to generate a T2 relaxation time to temperature model, according to various aspects of the present disclosure.

FIG. 13 is a graphical representation of an example relaxation model of T2 relaxation time to temperature, according to various aspects of the present disclosure.

FIG. 14 is a flow diagram for collecting T2 relaxation data to generate a heat map, according to various aspects of the present disclosure.

FIG. 15 is an example heat map generated from T2 relaxation data using a relaxation model, according to various aspects of the present disclosure.

The accompanying drawings are not intended to be drawn to scale. Corresponding reference characters indicate corresponding parts throughout the several views. For purposes of clarity, not every component may be labeled in every drawing. The exemplifications set out herein illustrate certain embodiments of the invention, in one form, and such exemplifications are not to be construed as limiting the scope of the invention in any manner.

### DETAILED DESCRIPTION

The following international patent applications are incorporated by reference herein in their respective entireties:
International Application No. PCT/US2020/018352, titled SYSTEMS AND METHODS FOR ULTRALOW FIELD RELAXATION DISPERSION, filed Feb. 14, 2020, now International Publication No. WO2020/168233;
International Application No. PCT/US2020/019530, titled SYSTEMS AND METHODS FOR PERFORMING MAGNETIC RESONANCE IMAGING, filed Feb. 24, 2020, now International Publication No. WO2020/172673;
International Application No. PCT/US2020/019524, titled PSEUDO-BIRDCAGE COIL WITH VARIABLE TUNING AND APPLICATIONS THEREOF, filed Feb. 24, 2020, now International Publication No. WO2020/172672;
International Application No. PCT/US2020/024776, titled SINGLE-SIDED FAST MRI GRADIENT FIELD COILS AND APPLICATIONS THEREOF, filed Mar. 25, 2020, now International Publication No. WO2020/198395;
International Application No. PCT/US2020/024778, titled SYSTEMS AND METHODS FOR VOLUMETRIC ACQUISITION IN A SINGLE-SIDED MRI SYSTEM, filed Mar. 25, 2020, now International Publication No. WO2020/198396;
International Application No. PCT/US2020/039667, title SYSTEMS AND METHODS FOR IMAGE RECONSTRUCTIONS IN MAGNETIC RESONANCE IMAGING, filed Jun. 25, 2020, now International Publication No. WO2020/264194;
International Application No. PCT/US2021/014628, titled MRI-GUIDED ROBOTIC SYSTEMS AND METHODS FOR BIOPSY, filed Jan. 22, 2021; and
International Application No. PCT/US2021/018834, titled RADIO FREQUENCY RECEPTION COIL NETWORKS FOR SINGLE-SIDED MAGNETIC RESONANCE IMAGING, filed Feb. 19, 2021;
International Patent Application No. PCT/US2021/021464, titled PHASE ENCODING WITH FREQUENCY SWEEP PULSES FOR MAGNETIC RESONANCE IMAGING IN INHOMOGENEOUS MAGNETIC FIELDS, filed Mar. 9, 2021; and
International Patent Application No. PCT/US2021/021461, titled PULSE SEQUENCES AND FREQUENCY SWEEP PULSES FOR SINGLE-SIDED MAGNETIC RESONANCE IMAGING, filed Mar. 9, 2021.

U.S. Patent Application Publication No. 2018/0356480, titled UNILATERAL MAGNETIC RESONANCE IMAGING SYSTEM WITH APERTURE FOR INTERVENTIONS AND METHODOLOGIES FOR OPERATING SAME, published Dec. 13, 2018, is also incorporated by reference herein in its entirety.

U.S. Provisional Patent Application No. 63/180,013, titled LOCALIZATION GUIDE AND METHOD FOR MRI GUIDED PELVIC INTERVENTIONS, filed Apr. 26, 2021, is also incorporated by reference herein in its entirety.

Before explaining various aspects of an MRI system and methods in detail, it should be noted that the illustrative examples are not limited in application or use to the details of construction and arrangement of parts illustrated in the accompanying drawings and description. The illustrative examples may be implemented or incorporated in other aspects, variations, and modifications, and may be practiced or carried out in various ways. Further, unless otherwise indicated, the terms and expressions employed herein have been chosen for the purpose of describing the illustrative examples for the convenience of the reader and are not for the purpose of limitation thereof. Also, it will be appreciated that one or more of the following-described aspects, expressions of aspects, and/or examples, can be combined with any one or more of the other following-described aspects, expressions of aspects, and/or examples.

In accordance with various aspects, an MRI system is provided that can include a unique imaging region that can be offset from the face of a magnet. Such offset and single-sided MRI systems are less restrictive as compared to traditional MRI scanners. In addition, this form factor can have a built-in or inherent magnetic field gradient that creates a range of magnetic field values over the region of interest. In other words, the inherent magnetic field can be inhomogeneous. The inhomogeneity of the magnetic field strength in the region of interest for the single-sided MRI system can be more than 200 parts per million (ppm). For example, the inhomogeneity of the magnetic field strength in the region of interest for the single-sided MRI system can between 200 ppm and 200,000 ppm. In various aspects of the present disclosure, the inhomogeneity in the region of interest can be greater than 1,000 ppm and can be greater than 10,000 ppm. In one instance, the inhomogeneity in the region of interest can be 81,000 ppm.

The inherent magnetic field gradient can be generated by a permanent magnet within the MRI scanner. The magnetic field strength in the region of interest for the single-sided MRI system can be less than 1 Tesla (T), for example. For example, the magnetic field strength in the region of interest for the single-sided MRI system can be less than 0.5 T. In other instances, the magnetic field strength can be greater than 1 T and may be 1.5 T, for example. This system can operate at a lower magnetic field strength as compared to typical MRI systems allowing for a relaxation on the RX coil design constraints and/or allowing for additional mechanisms, like robotics, for example, to be used with the MRI scanner. Exemplary MRI-guided robotic systems are further described in International Application No. PCT/US2021/014628, titled MRI-GUIDED ROBOTIC SYSTEMS AND METHODS FOR BIOPSY, filed Jan. 22, 2021, for example.

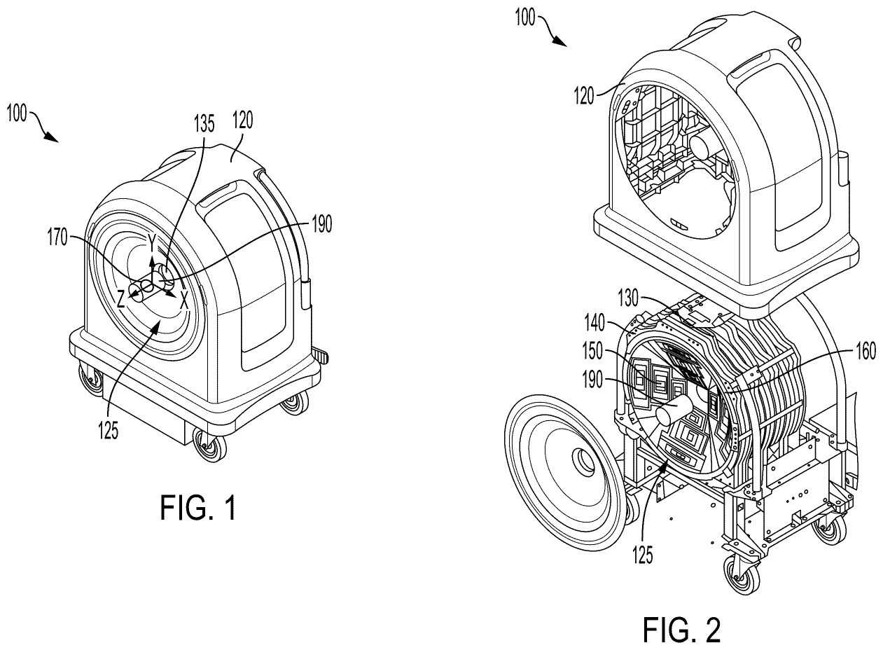

FIGS. 1-6 depict an MRI scanner 100 and components thereof. As shown in FIGS. 1 and 2, the MRI scanner 100 includes a housing 120 having a face or front surface 125, which is concave and recessed. In other aspects, the face of the housing 120 can be flat and planar. The front surface 125 can face the object being imaged by the MRI scanner. As shown in FIGS. 1 and 2, the housing 120 includes a permanent magnet assembly 130, an RF transmission coil (TX) 140, a gradient coil set 150, an electromagnet 160, and a RF reception coil (RX) 170. In other instances, the housing 120 may not include the electromagnet 160. Moreover, in certain instances, the RF reception coil 170 and the RF transmission coil 140 can be incorporated into a combined Tx/Rx coil array. In various instances, the MRI scanner 100 is a single-sided scanner and the various components, e.g. the permanent magnet assembly 130, the RF transmission coil (TX) 140, the gradient coil set 150, the electromagnet 160, and the RF reception coil (RX) 170, are positioned on the same side of the field of view.

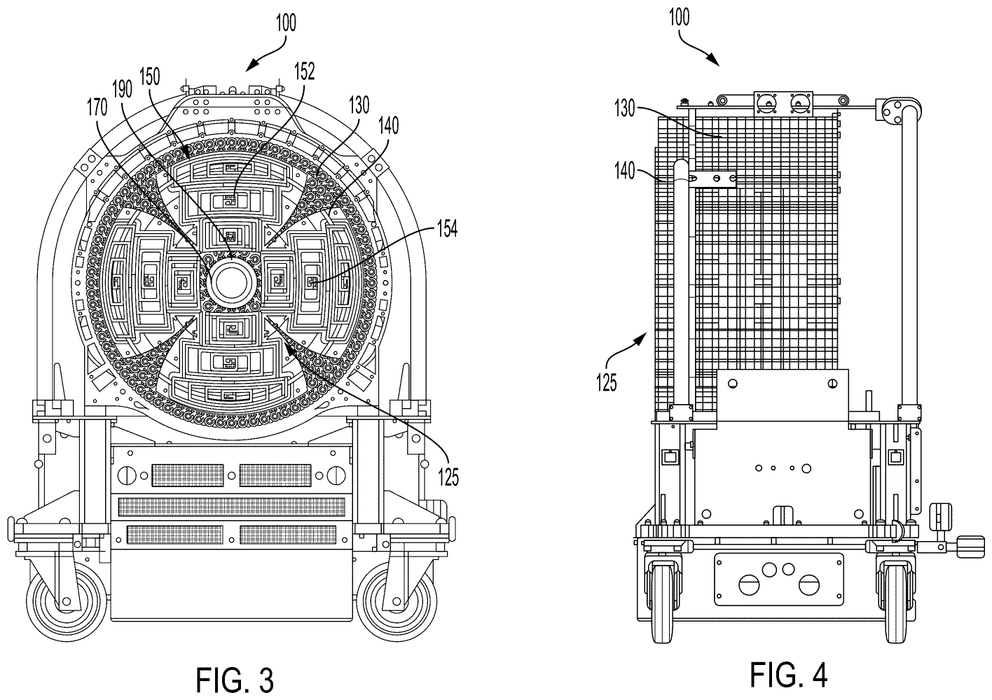

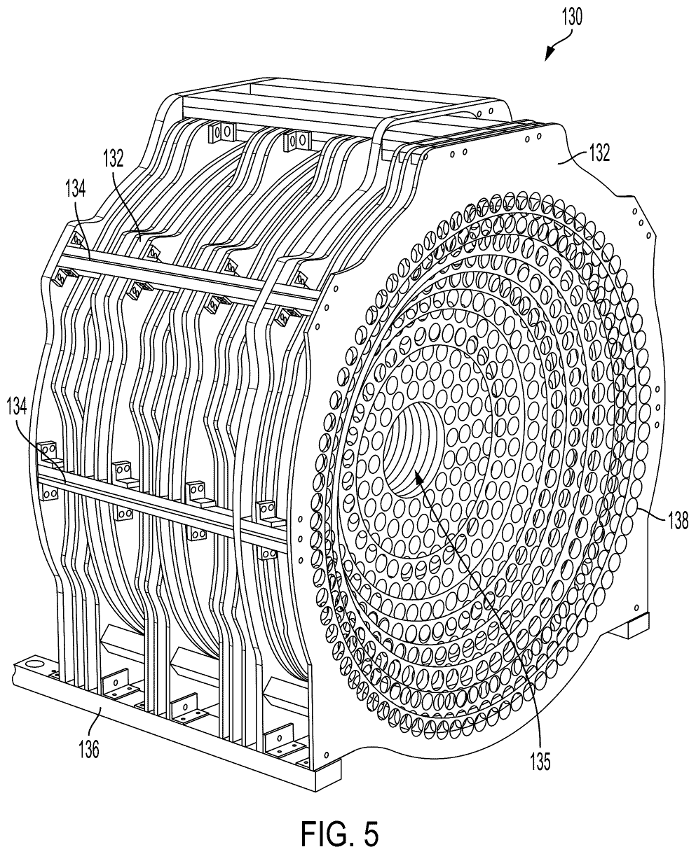

Referring primarily to FIGS. 3-5, the permanent magnet assembly 130 includes an array of magnets. The array of magnets forming the permanent magnet assembly 130 are configured to cover the front surface 125, or patient-facing surface, of the MRI scanner 100 (see FIG. 3) and are shown as horizontal bars in FIG. 4. The permanent magnet assembly 130 includes a plurality of cylindrical permanent magnets in a parallel configuration. Referring primarily to FIG. 5, the permanent magnet assembly 130 comprises parallel plates 132 that are held together by brackets 134. The system can be attached to the housing 120 of the MRI scanner 100 at a bracket 136. There can be a plurality of holes 138 in the parallel plates 132. The permanent magnet assembly 130 can include any suitable magnetic materials, including but not limited to rare-earth based magnetic materials, such as for example, Neodymium-based magnetic materials, for example.

The permanent magnet assembly 130 defines an access aperture or bore 135, which can provide access to the patient through the housing 120 from the opposite side of the housing 120. In other aspects of the present disclosure, the array of permanent magnets forming a permanent magnet assembly in the housing 120 may be bore-less and define an uninterrupted or contiguous arrangement of permanent magnets without a bore defined therethrough. In still other instances, the array of permanent magnets in the housing 120 may form more than one bore/access aperture therethrough.

In accordance with various aspects of the present disclosure, the permanent magnet assembly 130 provides a magnetic field BO in a region of interest 190 that is along the Z axis, shown in FIG. 1. The Z axis is perpendicular to the permanent magnet assembly 130. Stated differently, the Z axis extends from a center of the permanent magnet assembly 130 and defines a direction of the magnetic field BO away from the face of the permanent magnet assembly 130. The Z axis can define the primary magnetic field BO direction. The primary magnetic field BO can decrease along the Z axis, i.e. an inherent gradient, farther from the face of the permanent magnet assembly 130 and in the direction indicated with the arrow in FIG. 1

In one aspect, the inhomogeneity of the magnetic field in the region of interest 190 for the permanent magnet assembly 130 can be approximately 81,000 ppm. In another aspect, the inhomogeneity of the magnetic field strength in the region of interest 190 for the permanent magnet assembly 130 can be between 200 ppm to 200,000 ppm and can be greater than 1,000 ppm in certain instances, and greater than 10,000 ppm in various instances.

In one aspect, the magnetic field strength of the permanent magnet assembly 130 can be less than 1 T. In another aspect, the magnetic field strength of the permanent magnet assembly 130 can be less than 0.5 T. In other instances, the magnetic field strength of the permanent magnet assembly 130 can be greater than 1 T and may be 1.5 T, for example. Referring primarily to FIG. 1, the Y axis extends up and down from the Z axis and the X axis extends to the left and right from the Z axis. The X axis, the Y axis, and the Z axis are all orthogonal to one another and the positive direction of each axis is indicated by the corresponding arrow in FIG. 1.

The RF transmission coils 140 are configured to transmit RF waveforms and associated electromagnetic fields. The RF pulses from the RF transmission coils 140 are configured to rotate the magnetization produced by the permanent magnet 130 by generating an effective magnetic field, referred to as B1, that is orthogonal to the direction of the permanent magnetic field (e.g. an orthogonal plane).

Referring primarily to FIG. 3, the gradient coil set 150 includes two sets of gradient coils 152, 154. The sets of gradient coils 152, 154 are positioned on the face or front surface 125 of the permanent magnet assembly 130 intermediate the permanent magnet assembly 130 and the region of interest 190. Each set of gradient coils 152, 154 includes a coil portion on opposing sides of the bore 135. Referring to the axes in FIG. 1, the gradient coil set 154 may be the gradient coil set corresponding to the X axis, for example, and the gradient coil set 152 may be the gradient coil set corresponding to the Y axis, for example. The gradient coils 152, 154 enable encoding along the X axis and Y axis, as further described herein.

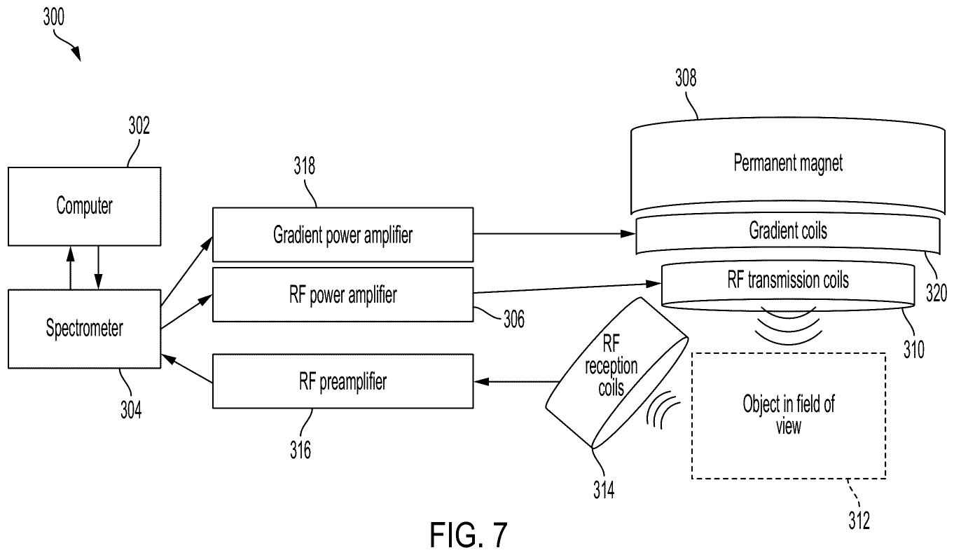

Referring now to FIG. 7, a control schematic for a single-sided MRI system 300 is shown. The single-sided MRI scanner 100 and/or components thereof (FIGS. 1-6) can be incorporated into the MRI system 300 in various aspects of the present disclosure. For example, the imaging system 300 includes a permanent magnet assembly 308, which can be similar to the permanent magnet assembly 130 (see FIGS. 2-5) in various instances. The imaging system 300 also includes RF transmission coils 310, which can be similar to the RF transmission coil 140 (see FIG. 3), for example. Moreover, the imaging system 300 includes RF reception coils 314, which can be similar to the RF reception coils 170 (see FIG. 3), for example. In various aspects, the RF transmission coils 310 and/or the RF reception coils can also be positioned in the housing of an MRI scanner and, in certain instances, the RF transmission coils 310 and the RF reception coils 314 can be combined into integrated Tx/Rx coils. The system 300 also includes gradient coils 320, which are configured to generate gradient fields to facilitate imaging of the object in the field of view 312.

The single-sided MRI system 300 also includes a computer 302, which is in signal communication with a spectrometer 304, and is configured to send and receive signals between the computer 302 and the spectrometer 304.

The main magnetic field BO generated by the permanent magnet 308 extends away from the permanent magnet 308 and away from the RF transmission coils 310 into the field of view 312. The field of view 312 contains an object that is being imaged by the MRI system 300.

During the imaging process, the main magnetic field BO extends into the field of view 312. The direction of the effective magnetic field (B1) changes in response to the RF pulses and associated electromagnetic fields from the RF transmission coils 310. For example, the RF transmission coils 310 are configured to selectively transmit RF signals or pulses to an object in the field of view, e.g. tissue. These RF pulses alter the effective magnetic field experienced by the spins in the sample (e.g. patient tissue). When the RF pulses are on, the effective field experienced by spins on resonance is solely the RF pulse, effectively canceling the static BO field. The RF pulses can be chirp or frequency sweep pulses, for example, as further described herein.

Moreover, when the object in the field of view 312 is excited with RF pulses from the RF transmission coils 310, the precession of the object results in an induced electric current, or MR current, which is detected by the RF reception coils 314. The RF reception coils 314 can send the excitation data to an RF preamplifier 316. The RF preamplifier 316 can boost or amplify the excitation data signals and send them to the spectrometer 304. The spectrometer 304 can send the excitation data to the computer 302 for storage, analysis, and image construction. The computer 302 can combine multiple stored excitation data signals to create an image, for example.

From the spectrometer 304, signals can also be relayed to the RF transmission coils 310 via an RF power amplifier 306, and to the gradient coils 320 via a gradient power amplifier 318. The RF power amplifier 306 amplifies the signal and sends it to RF transmission coils 310. The gradient power amplifier 318 amplifies the gradient coil signal and sends it to the gradient coils 320.

Systems and methods for effectively collecting nuclear magnetic resonance spectra and magnetic resonance images in inhomogeneous fields, such as with the single-sided MRI scanner 100 and system 300, for example, are described herein.

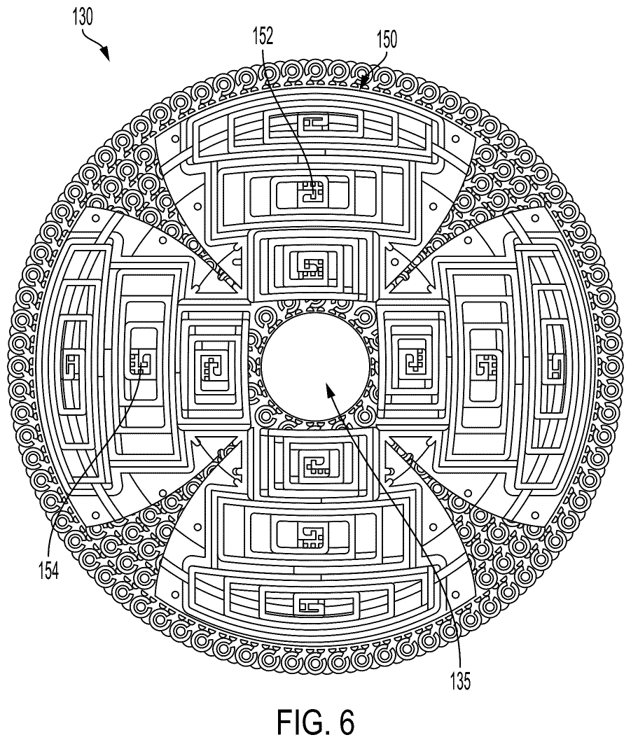

Imaging with a single-sided or open MRI presents many challenges. Typically, two sets of gradient coils (see FIG. 6) in single-sided systems are placed on the face of the permanent magnet assembly. As a result, the amplitude of the gradient will drop as one moves away from the face of the permanent magnet assembly. So, for a given array of phase encodes, the field of view will change as one moves along the axis of the permanent magnetic field BO. In other words, the pulsed gradient coils in a single-sided scanner have a small component along the direction of the permanent gradient.

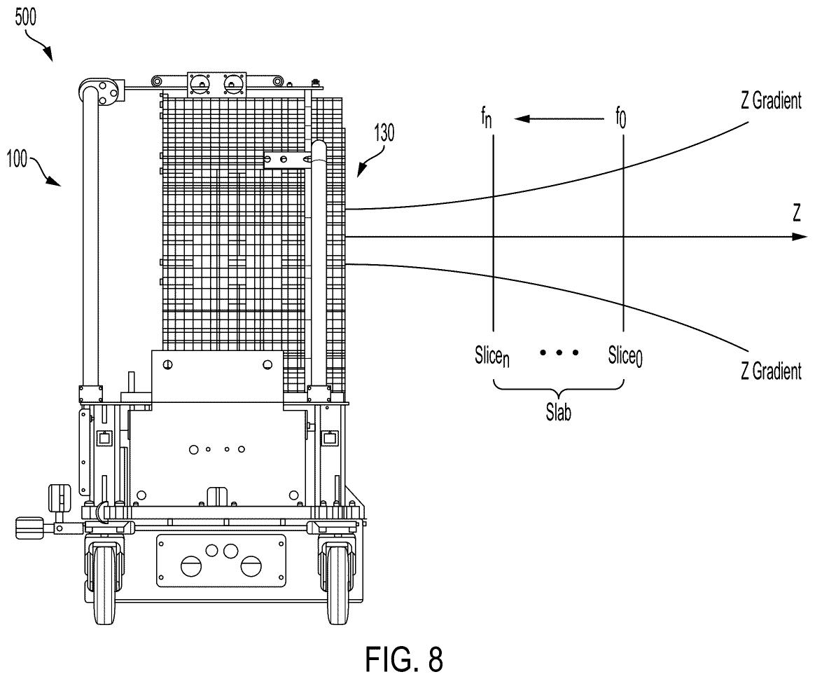

FIG. 8 is a schematic 500 of the magnetic field gradient along the Z axis for the MRI scanner 100. The permanent magnet 130 has an inherent gradient along the Z axis. The strength of the Z gradient decreases as one moves away from the permanent magnet 130. The Z gradient can be seen in the schematic bending away as one moves away from the permanent magnet causing the strength of the gradient to decrease. The MRI scanner 100 images multiple slices to create a slab. Each slice is excited for imaging at a different frequency. The lower frequencies excite tissue for slices farther away from the permanent magnet and higher frequencies excite the tissue in slices closer to the magnet. In the schematic, the slab or axial image is made of multiple slices going from Sliceo to Slicen. Each slice has a corresponding frequency f₀ to fₙ, where f₀ is a frequency that is smaller than fₙ.

In accordance with various aspects of the present disclosure, it is possible to compensate for added phase by applying a phase encode during a frequency sweep, or chirped, excitation pulse. A frequency sweep pulse can affect spins at different frequencies at different times during a pulse. This means that it is also possible to impart different amounts of phase to different frequencies by applying a phase encode during an excitation pulse. The spins excited at the beginning of the pulse can accumulate more phase than the spins excited at the end of the pulse, which can accumulate little phase.

In accordance with various aspects, if the spins further from the permanent magnet are excited first, and if a phase encode is applied during the frequency sweep excitation pulse, then those farther away spins can accumulate more phase than the spins closer to the permanent magnet, which can be excited last. This can invert the usual way spins accumulate phase from a surface gradient coil, allowing one to counter the normal variation in gradient strength along the Z axis. By precisely tuning the amount of phase accumulated during the frequency sweep excitation and during a subsequent phase encode, it is possible to apply an even amount of phase to the X-Y plane along the Z axis of the permanent magnet.

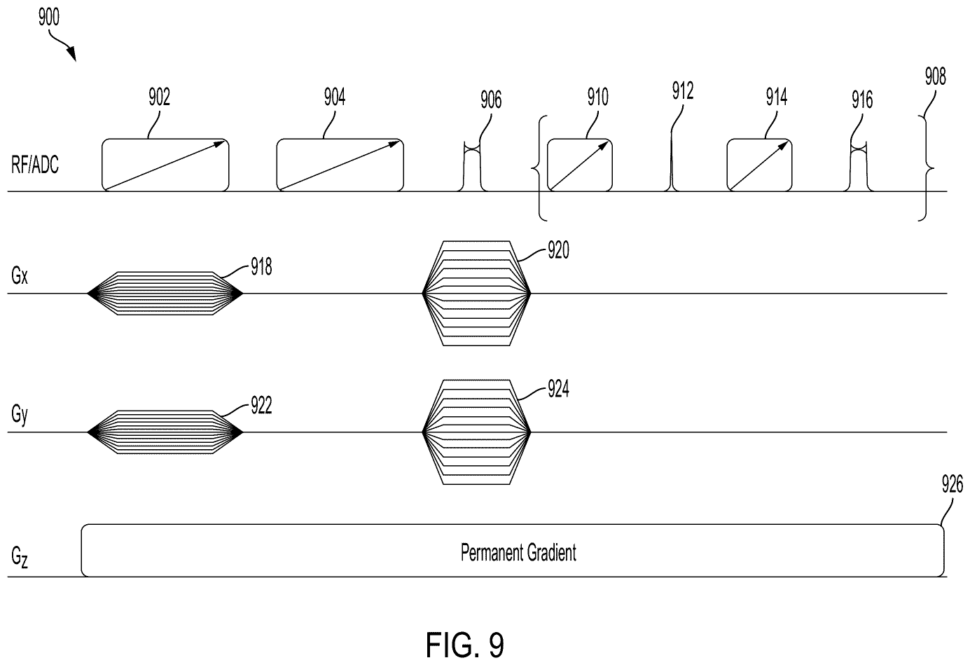

FIG. 9 shows a pulse sequence 900 that is configured to compensate for the varying field of view in slices along the Z axis produced by surface gradient coils (see for example gradient coils 152, 154 in FIG. 6). This compensation is achieved with phase encoding applied during a frequency sweep excitation pulse. In various instances, the frequency sweep pulses described herein are chirp or chirped pulses having a linear frequency sweep. A chirped excitation pulse can define a linear frequency sweep from low to high. Other monotonic low-to-high frequency increases are also contemplated. The low frequencies excite tissue farther from the permanent magnet assembly (see, e.g. the permanent magnet assembly 130 in FIG. 2) and the high frequencies excite tissue closer to the permanent magnet assembly, so by the end of the pulse, slices further from the magnet will have been phase encoded for more time, compensating for the gradient being weaker. The first pulse 902 in the pulse sequence is a frequency sweep excitation pulse 902, with the chirp frequency swept direction set from low to high. The gradients in the X and Y directions begin to dephase 918 and 922, respectively, and are refocused by the second pulse 904 in the pulse sequence. The gradient in Z is constant during the entire pulse sequence. The second pulse 904 is a refocusing pulse that refocuses the X and Y gradients. After a second pulse 904, a spectral echo 906 occurs where the X and Y gradients dephase 920 and 924, respectively. After the spectral echo 906, the signal is then read out with a chirped echo train 908. The chirped echo train 908 comprises a third pulse 910, a spin echo 912, a fourth pulse 914, and a spectral echo 916. In one aspect, the third pulse 910 may be a second refocusing pulse and the fourth pulse 914 may be a second excitation pulse.

In this implementation, the changing field of view is overcompensated during the excitation pulse and then balanced with the phase encode. The amount of phase accumulated during the frequency sweep needs to be precisely tuned to apply an even amount of phase to the X-Y plane of the slices being imaged. Stated another way, the amount of phase in each slice needs precisely tuned to account for the changing field of view. Stated yet another way, the scale of the object in each slice needs to be adjusted so that all the slices have the object scaled the same. For example, the tuning can be performed by adjusting the power of the gradient pulse applied during the frequency sweep pulse while collecting a 2D image along the X-Z or Y-Z axes. The gradient power can be increased until the size of the object does not change along the Z axis. Then, the slices can be combined into a high quality slab image without any blurring occurring from the combination.

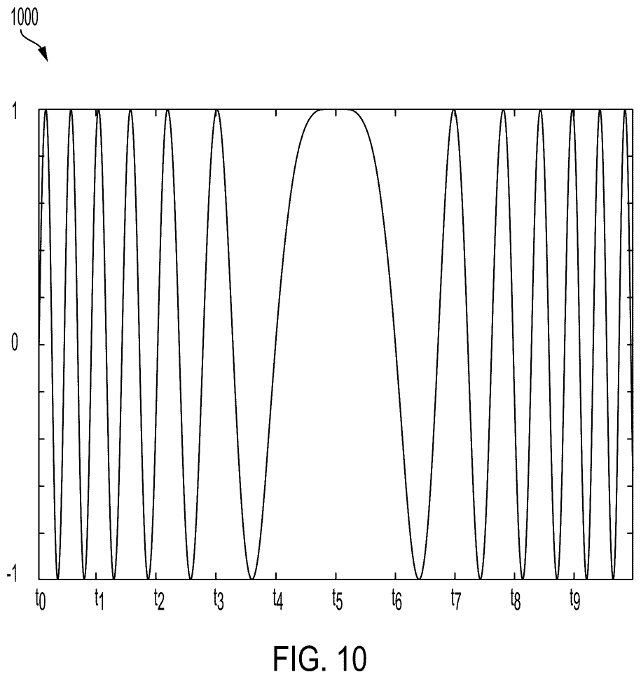

FIG. 10 shows a representative graph 1000 of a sweeping frequency pulse or chirp pulse, where the swept direction is set from low to high. A chirped excitation pulse, with the swept direction set from low to high, is an example of a frequency sweep excitation pulse. The frequency of a chirp pulse with the swept direction set from low to high begins at a low frequency and the frequency increases through time for the duration of the pulse. The pulse can begin at the lowest frequency desired and ends once the maximum desired frequency is reached. The pulse frequency in the graph 1000 can be a negative-to-positive frequency offset to the baseband frequency. In other words, the frequency sweeps from negative to positive plus the baseband frequency. For example, for a frequency sweep of +/−100 KHz, the sweep is from the baseband frequency less 100 KHz to the baseband frequency plus 100 KHz.

The frequency of a chirp pulse can vary from a minimum (lowest) desired frequency to a maximum (highest) desired frequency. The sweep rate of the pulse is the difference between the highest frequency and lowest frequency in the pulse divided by the time required to go between the highest frequency and the lowest frequency. In one aspect, the frequency range that is covered by the sweeping frequency pulses used in the sweeping frequency pulse sequence 900 may be from-20 KHz to 20 KHz, i.e. a 40 KHz range, with a center frequency that varies slab to slab. For example, a slab could be centered at 2.62 MHz, 2.75 MHz, 2.65 MHz, 2.72 MHZ, 2.79 MHZ, 2.69 MHZ, and so on. For a slab centered at 2.62 MHz, the chirp pulse would sweep from 2.60 MHz to 2.64 MHZ, i.e. a 40 KHz range. In other aspects of the present disclosure, bandwidths as low as 10 KHz to as high as 200 KHz may be used in the frequency sweep pulse. Moreover, the sweep range can be less than 40 KHz in various instances.

Referring again to FIG. 8, f₀ can corresponds to the lowest frequency of the chirp pulse and fₙ can corresponds to the highest frequency of the chirp pulse. The chirp pulse excites tissue farther away from the permanent magnet assembly first, such as tissue at the location of sliceo, and excites the tissue close to the permanent magnet assembly later, such as the tissue at the location of slicen. Stated another way, adjacent slices comprise a proximal slice and a distal slice, where the proximal slice is positioned closer to the magnetic imaging apparatus than the distal slice, and a target in the distal slice is excited before a target in the proximal slice. The frequency range of the chirp pulse may correspond to the slices of the slab being imaged.

Referring again to FIG. 9, the first pulse 902 is a chirped excitation pulse with the swept direction set from low to high. This pulse excites tissue in slices farther from the permanent magnet assembly before exciting tissue in slices closer to the permanent magnet assembly. By phase encoding during the chirped excitation there is a different amount of phase accumulated at different frequencies. Specifically, the slices farther away from the permanent magnet assembly accumulate more phase than slices closer to the permanent magnet assembly. Stated another way, the target in the slices that are more distal from the permanent magnet assembly accumulate more phase than the target in the slices that are more proximal to the permanent magnet assembly. Phase encoding during the frequency sweep excitation pulse along with the tuning of the phase accumulated in each slice can account for the phases in each slice and keep the echo from drifting outside of the acquisition window. After accounting for the changing field of view in slices along the Z axis, the slices can be combined into a slab to produce a high quality axial image, where the scale of the object in each slice is the same size.

Several methods for measuring temperatures using magnetic resonance exist. The most common method is using chemical shift. The chemical shift of water will change depending on the temperature, which allows one to monitor temperature by monitoring how the chemical shift changes. This method only works for voxels with exchanging protons, like water. Other molecules, like fat, will not have a chemical shift that changes with temperature. Monitoring the temperature of fats is typically done by measuring the relaxation time of the tissue. As the temperature changes, so too does the relaxation rate. Relaxation maps can be collected and then converted to a temperature map with a calibration for that specific type of tissue.

In a chemical shift based method, frequency changes from the chemical shift are small at low magnetic fields. If the main magnetic field is inhomogeneous, then the frequency spread from the inhomogeneity will obscure the chemical shift changes due to the temperature. This issue makes chemical shift based methods unsuitable for low magnetic field, single-sided MRI systems. A relaxation based temperature measurement method is needed for low field single-sided systems.

As described above, within inhomogeneous magnetic fields, frequency shifts from chemical shift can be undetectable as measuring them would require a field homogeneous enough to resolve the chemical shift differences. Temperature changes can instead be monitored by using relaxation data, such as T2 relaxation times. The temperature of tissue will affect the exchange rates of bound water in the body and change the overall motion of spins within the voxel, which subsequently changes the T2 relaxation time. These T2 relaxation times can then be converted to temperature with a model that correlates T2 relaxation to temperature. A model can be constructed by calibrating a specific tissue type through measuring its T2 relaxation time at different temperatures. Each tissue type requires a different model. By fitting the T2 relaxation times to a model that incorporates changes in rotation diffusion and changes in exchange times, the temperature of each voxel can be estimated, resulting in a low field temperature map.

MRI scanner 100 (FIG. 1) is an example of a single-sided MRI system that can perform the method described above. The single-sided MRI system would comprise a housing comprising a face, wherein a first axis extends through the face into a field of view. The single sided MRI system would further comprise an array of permanent magnets positioned in the housing, wherein an inherent gradient magnetic field extends from the array of permanent magnets relative to the first axis into the field of view. The single sided MRI system would further comprise a gradient coil set, a radio frequency coil, a power circuit, a memory storing a relaxation model for a tissue type, and a control circuit in signal communication with the gradient coil set, the radio frequency coil, and the memory. The control circuit would be configured to obtain a T2 data set from the radio frequency coil related to a structure positioned in the field of view, wherein the structure corresponds to the tissue type. For example, the structure can be comprised of tissue of the tissue type. The control circuit would be further configured to generate a T2-weighted image of the structure, and convert the T2-weighed image of the structure to a heat map based on the relaxation model for the tissue type.

A single-sided MRI system may provide many benefits to guided thermal ablation of soft tissues in vivo. For example, the MRI system is open and provides better access to patients than standard, closed MRI systems. Additionally, the single-sided MRI system may be more cost effective than the closed MRI systems. In various instances, a single-sided MRI system may provide better outcomes to thermal ablation due to allowing better access to the patient and the thermal ablation area as well as being able to provide live temperature maps of the thermal ablation area. Comparatively, conventional high-field MRI systems are often clinically impractical for thermal therapy applications due to their high cost, operational complexities, and difficult to access form factors.

Since this method for measuring temperatures using magnetic resonance is for a system having an inhomogeneous magnetic field resulting from the single-sided geometry of the apparatus, the echo train sequence can be collected with chirped pulses broad enough to excite and refocus efficiently in the field. In one aspect, the pulse sequence may have phase encodes along two axes but no additional encoding during the echo train. In an alternative aspect, the pulse sequence may have phase encodes along two axes and additional encoding during the echo train. In another aspect, one of the imaging axes may be spatiotemporally encoded while the other is encoded with phase encodes. In yet another aspect, both the x and y axes could be spatiotemporally encoded. The chirped pulses may be generated by the radio frequency coil and the phase encoding may be applied by the gradient coil set.

In an alternative aspect, the method could be applied by a homogeneous magnetic field.

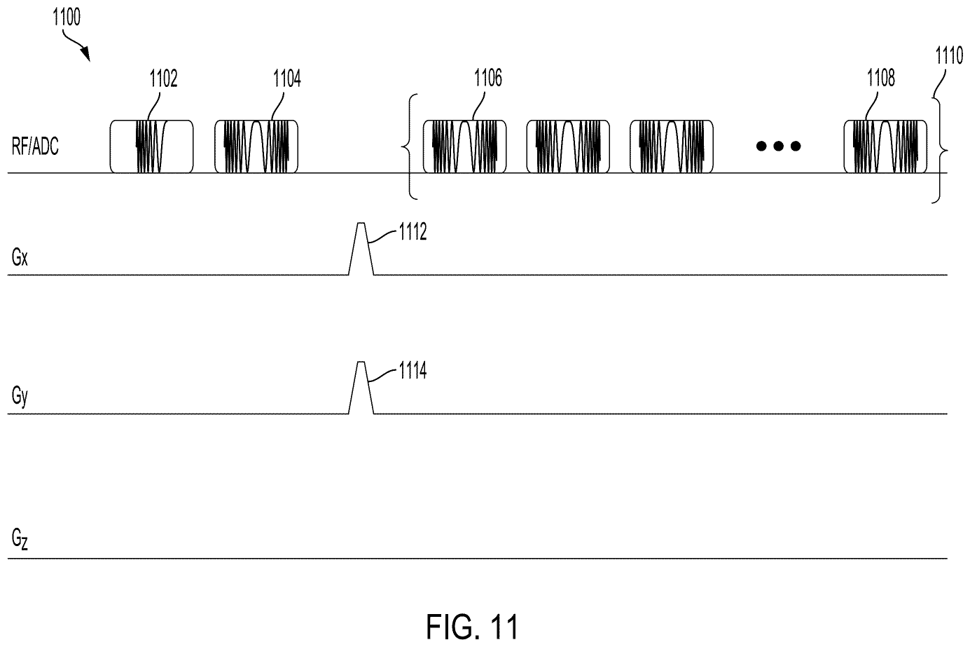

FIG. 11 shows a diagram of a pulse sequence 1100 that can be used to collect T2 relaxation times of a structure in the field of view of the MRI system. The pulse sequence can be used with a single-sided open MRI system of FIG. 1 utilizing a low magnetic field, for example. Similar to the pulse sequence 900 (FIG. 9), the first pulse in the pulse sequence 1100 is a frequency sweep excitation pulse 1102, with the chirp frequency swept direction set from low to high. The gradient in Z is constant during the entire pulse sequence. After the second pulse 1104, phase encoding 1112 and 1114 is applied along the X and Y gradients, respectively. Then, a plurality of sweeping frequency pulses 1110 can be transmitted by the radio frequency coil. In FIG. 11, the sweeping frequency pulse 1106 is the first pulse of the plurality of sweeping frequency pulses 1110 and the sweeping frequency pulse 1108 is the last pulse in the plurality of sweeping frequency pulses 1110.

The plurality of sweeping frequency pulses 1110 comprises an echo train sequence that is received by the radio frequency coil. In some aspects, the radio frequency coil can be a single unit that both transmits and receives. In other aspects, the radio frequency coil can comprise a set of at least two coils, where at least one coil transmits and at least one coil receives. Each sweeping frequency pulse produces an echo and each echo can have an echo time from approximately two milliseconds to approximately twenty milliseconds. The echo train sequence may comprise anywhere from approximately ten echoes to approximately 100 echoes. In an alternate aspect, there could be more than 100 or less than ten echoes in the echo train sequence. The bandwidth of the sweeping frequency pulses may be between approximately 10 KHz to approximately 200 KHz.

T2 data can be calculated from each echo in the echo train sequence. In one aspect, the T2 data set can be generated based on the plurality of sweeping frequency pulses. In another aspect, the T2 data set could be generated based on the plurality of sweeping frequency pulses and phase encoding during the plurality of sweeping frequency pulses. T2 relaxation times can be determined by fitting the echo amplitudes against the echo time to a monoexponential decay function.

The method for measuring temperatures in vivo using magnetic resonance comprises first collecting a calibration for the kind of tissue that is being measured. A calibration requires that excised tissue or a phantom designed to mimic the tissue has its T2 relaxation time measured at various temperatures. The response of the relaxation time to temperature is then fit to a model. Each tissue type can have a unique model.

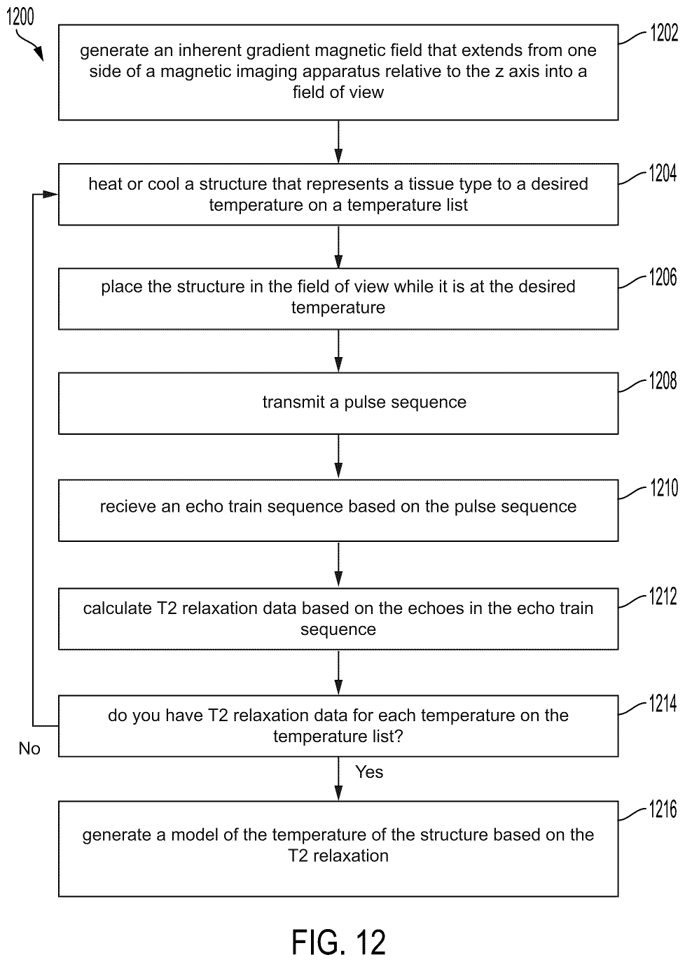

FIG. 12 shows a flow diagram 1200 for collecting calibration data for a particular tissue type. The calibration data can then be used to generate a T2 relaxation time to temperature model for the particular tissue type. T2 relaxation times are measured for the tissue type at various temperatures. For example, a structure comprised of the tissue type can be excised tissue or can be a phantom that is designed to mimic the tissue type. At block 1202, an MRI system, for example MRI scanner 100 (FIG. 1), generates an inherent gradient magnetic field that extends from one side of a magnetic imaging apparatus relative to the z axis into a field of view. At block 1204, the structure is heated or cooled to a desired temperature on a temperature list. The temperature list can include various temperatures that are possible for the tissue type to reach. Stated another way, the temperature list can encompass a plurality of temperatures that are anticipated for the tissue type of the structure. At block 1206, the structure is placed in the field of view while it is at the desired temperature. At block 1208, the MRI system transmits a pulse sequence, such as pulse sequence 1100 (FIG. 11) for example. At block 1210, the MRI system receives an echo train sequence based on the pulse sequence. At block 1212, the MRI system calculates T2 relaxation data based on the echoes in the echo train sequence. At block 1214, the MRI system checks to see if each temperature on the temperature list has a corresponding T2 relaxation time. If there is not T2 relaxation data for each temperature on the temperature list, then the system proceeds to block 1204, and the structure is heated or cooled to another temperature on the temperature list. The foregoing data acquisition loop can continue until each temperature on the temperature list has T2 relaxation data. When each temperature has corresponding T2 relaxation data, the system proceeds to block 1216. At block 1216, a model is generated for the temperature of the structure based on the T2 relaxation data at the temperatures on the temperature list. Additional models can be obtained for different tissue types.

In an example calibration method, phantoms were made from bottles filled with honey, which were used for the structure. The calibration step was performed on the phantoms by subjecting the phantoms to controlled heating in a water bath to various temperatures. The phantoms were then placed in a receive coil that was surrounded by insulation. Once placed in the insulated receive coil, the bulk T2 relaxation time of the phantom was obtained with the MRI apparatus, and the temperature of the phantom was also immediately measured with a FLIR-E6390 thermal camera. Once the T2 relaxation time and the corresponding temperature was measured, the phantom was taken out of the receive coil and heated in a water bath to another desired temperature. The process was repeated to obtain a successful measurements of T2 relaxation time for each desired temperature. The T2 relaxation times were determined by fitting the echo amplitudes against the echo time to a monoexponential decay function.

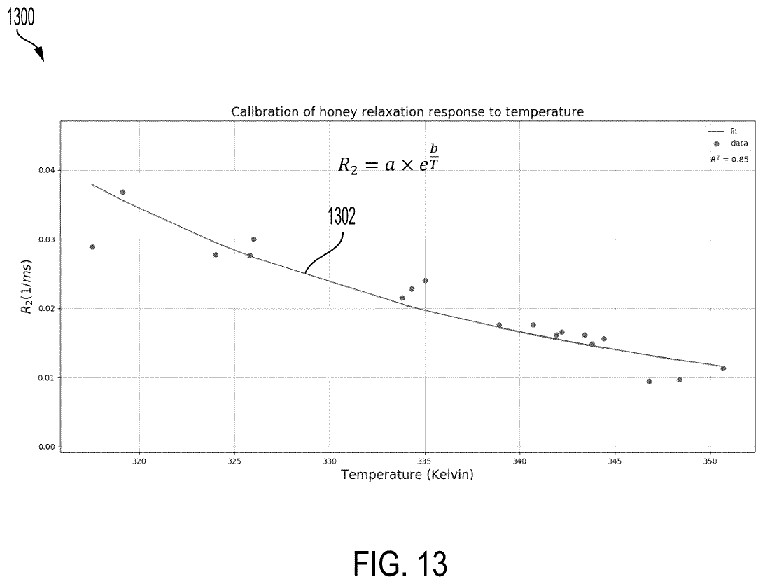

T2 relaxation times and the corresponding temperatures measurements for the example calibration method are shown in a graphical representation 1300 in FIG. 13, where the phantoms (bottles filled with honey) were used. The y-axis of the graphical representation 1300 is R₂, where R₂ is the inverse of the T2 relaxation time. Stated another way R₂ is 1/T2. In one aspect, the R₂ times were found by fitting the signal amplitude decay in an echo train to a monoexponential function. An example model associating relaxation time to temperature is shown by line 1302 of the graphical representation 1300. Line 1302 is described by the following equation:

$${R}_{2} = a \times {e}^{\frac{b}{T}} ,$$

where the fitting parameters were log (a)=−15.8±2.3 and b=3976±770, R₂ was 1/T2, and T was temperature in Kelvin. The foregoing equation corresponds to the line 1302 and is an example model of the relaxation time to temperature relationship for a particular sample.

In various instances, the relaxation time to temperature relationship can correspond to a monoexponential model. For example, in the example calibration method and associated data set shown in FIG. 13, T2 relaxation time changed monotonically with temperature with the phantom and increased as the temperature increased. This is consistent with the viscosity of honey decreasing as it is heated, which can be clearly seen by observing how honey flows more readily at higher temperatures. Furthermore, in various aspects of the present disclosure, to generate the model and relationship shown in the equation above, the product of the Larmor frequency and correlation time can be assumed to be much less than one, making R₂ proportional to the rotational correlation time.

In an alternative aspects of the present disclosure, other equations could be used to fit the T2 relaxation time and temperature data.

Once the calibration data for the relevant tissue types are completed and a model for each tissue type is generated, temperature maps or heat maps for the relevant tissue types can be collected. To generate the temperature maps, a T2 map or T2 weighted image can be obtained with a 3D echo train sequence, as further described herein. The 3D echo train sequence can include phase encodes along two axes but no additional encoding blips during the echo train. Such a pulse sequence results in a data set in which each echo is a separate image with different T2 weighting. In various instances, each echo can be reconstructed into a separate image. The series of images can be converted into a single T2 map by fitting the pixel intensity to an exponential decay against echo time (e.g. monoexponential function). Each pixel that has signal will produce a T2 time from this fit. The T2 map that results from this fit can then be converted to a temperature map with the calibration, such as the calibration equation described above for a particular structure. The calibration equation can map T2 relaxation times to a temperature, provided that the tissue type is known.

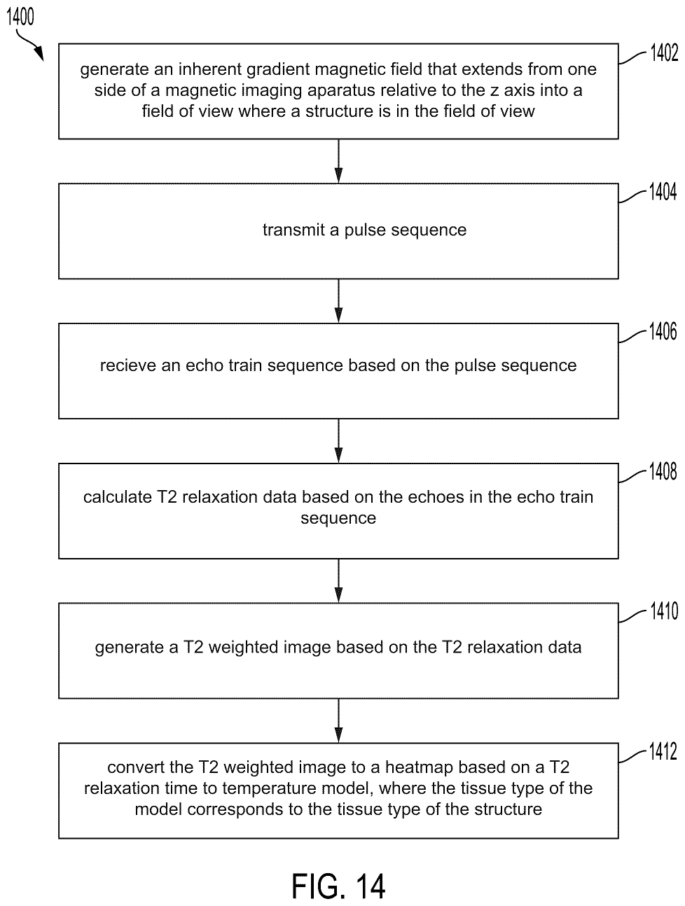

FIG. 14 shows a flow diagram 1400 for collecting T2 relaxation data and using that data to generate a heat map based on a T2 relaxation time to temperature model, which can be obtained using the calibration method further described herein. At block 1402, an MRI system (for example MRI scanner 100 (FIG. 1)), generates an inherent gradient magnetic field that extends from one side of a magnetic imaging apparatus relative to the z axis into a field of view where a structure is placed in the field of view. At block 1404, the MRI system transmits a pulse sequence, such as pulse sequence 1100 (FIG. 11) for example. At block 1406, the MRI system receives an echo train sequence based on the pulse sequence. In one aspect, the echo train sequence could be a 3D echo train sequence. At block 1408, the MRI system calculates T2 relaxation data based on the echoes in the echo train sequence. At block 1410, the MRI system generates a T2 weighted image based on the T2 relaxation data. At block 1412, the MRI system converts the T2 weighted image to a heatmap based on a T2 relaxation time to temperature model. The tissue type of the structure is the same as the tissue type for the T2 relaxation time to temperature model. In one aspect, the tissue type of the structure can be input manually so that the correct model can be used. In another aspect, the tissue type of the structure could be calculated automatically based on a predetermined tissue type model. For example, structural information from the predetermined model can be mapped onto the image of the structure to determine the tissue type.

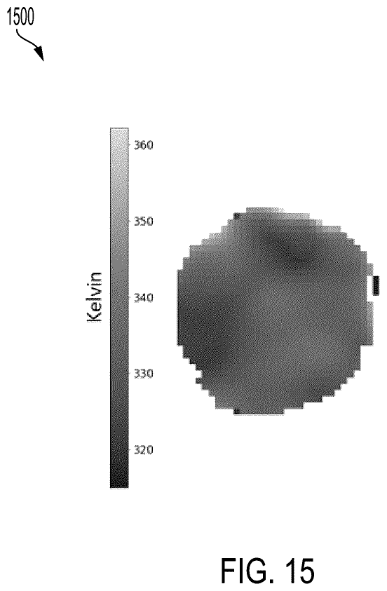

To generate an example temperature map 1500, shown in FIG. 15, the structure used for the calibration data was heated again and then placed in the insulated coil to be imaged. A 3D single-point imaging scan with an echo train was used to collect a T2-weighted image, or T2 map. Each echo was used to generate a separate image, and the intensity of each pixel was fit to a monoexponential decay function against echo time. From these fits, a T2 map for the heated structure was generated and then converted to the temperature map 1500 with the example calibration model.

The T2 map used to generate the heat map 1500 was collected with a single-sided open MRI system with a permanent gradient and a low field MRI. For example, the main magnetic field strength can operate at 58-74 mT. An example method to collect a T2 map is to use a 3D single point imaging sequence.

In alternative aspects, other MRI systems could be used to collect the T2 map that is used to generate a heat map.

The temperature maps obtained with the foregoing methods show significant temperature gradients that are consistent with the thermal camera measurements. Due to the low magnetic field and form factor of the low-field, single sided MRI apparatus, the foregoing relaxation based MR thermometry can be integrated with thermal ablation devices to improve guidance and obtain better treatment outcomes in certain instances.

### Examples

Various aspects of the subject matter described herein are set out in the following numbered examples.

Example 1-A magnetic imaging apparatus comprising a housing comprising a face, wherein a first axis extends through the face into a field of view. The magnetic imaging apparatus further comprising an array of permanent magnets positioned in the housing, wherein an inherent gradient magnetic field extends from the array of permanent magnets relative to the first axis into the field of view. The magnetic imaging apparatus further comprising a gradient coil set, at least one radio frequency coil, a power circuit, a memory storing a relaxation model for a tissue type, and a control circuit in signal communication with the gradient coil set, the at least one radio frequency coil, the power circuit, and the memory. The control circuit is configured to obtain a T2 data set related to a structure positioned in the field of view, wherein the structure corresponds to the tissue type. The control circuit is further configured to generate a T2-weighted image of the structure, and convert the T2-weighed image of the structure to a heat map based on the relaxation model for the tissue type.

Example 2-The magnetic imaging apparatus of Example 1, wherein the relaxation model comprises a monoexponential model.

Example 3-The magnetic imaging apparatus of Examples 1 or 2, wherein the gradient coil and the at least one radio frequency coil are coupled to the power circuit.

Example 4-The magnetic imaging apparatus of Examples 1, 2, or 3, wherein the control circuit is further configured to transmit a pulse sequence comprising a plurality of sweeping frequency pulses.

Example 5-The magnetic imaging apparatus of Example 4, wherein the T2 data set is generated from the plurality of sweeping frequency pulses.

Example 6-The magnetic imaging apparatus of Examples 4 or 5, wherein each sweeping frequency pulse produces an echo comprising a duration between 2 milliseconds and 20 milliseconds.

Example 7-The magnetic imaging apparatus of Examples 4, 5, or 6, wherein the plurality of sweeping frequency pulses produces between 10 echoes and 100 echoes.

Example 8-The magnetic imaging apparatus of Examples 4, 5, 6, or 7, wherein each sweeping frequency pulse comprises a bandwidth between 10 KHz and 200 KHz.

Example 9-The magnetic imaging apparatus of Examples 1, 2, 3, 4, 5, 6, 7, or 8, wherein the radio frequency coil is configured to transmit pulses having a frequency between 1 Megahertz and 21 Megahertz.

Example 10-The magnetic imaging apparatus of Examples 1, 2, 3, 4, 5, 6, 7, 8, or 9, wherein the magnetic field strength in the field of view is less than 1 Tesla.

Example 11-The magnetic imaging apparatus of Examples 1, 2, 3, 4, 5, 6, 7, 8, 9, or 10, wherein the inhomogeneity of the magnetic field in the field of view is between 200 ppm and 200,000 ppm.

Example 12-The magnetic imaging apparatus of Examples 1, 2, 3, 4, 5, 6, 7, 8, 9, 10, or 11, wherein the relaxation model is generated from calibration data comprising a plurality of T2 relaxation data for different temperatures for the tissue type.

Example 13-The magnetic imaging apparatus of Examples 1, 2, 3, 4, 5, 6, 7, 8, 9, 10, 11, or 12, wherein the magnetic imaging apparatus is a single-sided magnetic imaging apparatus, and wherein the housing and the gradient coil set are positioned on a first side of field of view.

Example 14-The magnetic imaging apparatus of Examples 1, 2, 3, 4, 5, 6, 7, 8, 9, 10, 11, 12, or 13, further comprising a user input device configured to receive the tissue type of the structure.

Example 15-The magnetic imaging apparatus of Examples 1, 2, 3, 4, 5, 6, 7, 8, 9, 10, 11, 12, or 13, further comprising an automated tissue type recognition module configured to identify a tissue type model corresponding to the structure, and register the tissue type model with the structure.

Example 16-A single-sided magnetic imaging apparatus, comprising a housing comprising an array of permanent magnets, wherein an inherent gradient magnetic field extends from the array of permanent magnets relative to a first axis into a field of view, wherein the field of view is adjacent to the housing. The single-sided magnetic imaging apparatus further comprises a radio frequency coil, a power circuit coupled to the radio frequency coil, a memory storing relaxation models for tissue at different temperatures, and a control circuit. The control circuit is in signal communication with the radio frequency coil, the power circuit, and the memory. The control circuit is configured to transmit a waveform sequence to the radio frequency coil to produce an echo train sequence, obtain a T2 data set related to a structure positioned in the field of view, generate a T2-weighted image of the structure, and convert the T2-weighed image of the structure to a heat map based on the relaxation models stored in the memory.

Example 17-The single-sided magnetic imaging apparatus of Example 16, wherein the relaxation models comprise a monoexponential model.

Example 18-The single-sided magnetic imaging apparatus of Examples 16 or 17, wherein the echo train sequence comprises phase encodes.

Example 19-The single-sided magnetic imaging apparatus of Examples 16, 17, or 18, wherein the T2 data set is generated based on the echo train sequence.

Example 20-The single-sided magnetic imaging apparatus of Examples 16, 17, 18, or 19, wherein the echo train sequence comprises echoes comprising a duration between 2 milliseconds and 20 milliseconds.

Example 21-The single-sided magnetic imaging apparatus of Examples 16, 17, 18, 19, or 20, wherein the echo train sequence comprises between 10 echoes and 100 echoes.

Example 22-The single-sided magnetic imaging apparatus of Examples 16, 17, 18, 19, 20, or 21, wherein the echo train sequence is produced by a plurality of sweeping frequency pulses comprising a bandwidth between 10 KHz and 200 KHz.

Example 23-The single-sided magnetic imaging apparatus of Examples 16, 17, 18, 19, 20, 21, or 22, wherein the radio frequency coil is configured to transmit pulses having a frequency between 1 Megahertz and 21 Megahertz.

Example 24-The single-sided magnetic imaging apparatus of Examples 16, 17, 18, 19, 20, 21, 22, or 23, wherein the magnetic field strength in the field of view is less than 1 Tesla.

Example 25-The single-sided magnetic imaging apparatus of Examples 16, 17, 18, 19, 20, 21, 22, 23, or 24, wherein the inhomogeneity of the magnetic field in the field of view is between 200 ppm and 200,000 ppm.

Example 26-The single-sided magnetic imaging apparatus of Examples 16, 17, 18, 19, 20, 21, 22, 23, 24, or 25, wherein the relaxation models are generated from calibration data comprising a plurality of T2 data at different temperatures for different tissue types.

Example 27-The single-sided magnetic imaging apparatus of Examples 16, 17, 18, 19, 20, 21, 22, 23, 24, 25, or 26, further comprising a gradient coil set positioned in the housing, wherein the gradient coil set, the radio frequency coil, and the housing are positioned on a first side of the field of view.

Example 28-The single-sided magnetic imaging apparatus of Examples 16, 17, 18, 19, 20, 21, 22, 23, 24, 25, 26, or 27, wherein the radio frequency coil comprises a radio frequency transmission coil, and wherein the single-sided magnetic imaging apparatus further comprises a radio frequency reception coil.

Example 29-The single-sided magnetic imaging apparatus of Examples 16, 17, 18, 19, 20, 21, 22, 23, 24, 25, 26, 27, or 28, further comprising a user input device configured to receive the tissue type of the structure, wherein each relaxation model corresponds to a tissue type.

Example 30-The single-sided magnetic imaging apparatus of Examples 16, 17, 18, 19, 20, 21, 22, 23, 24, 25, 26, 27, or 28, further comprising an automated tissue type recognition module configured to identify a tissue type model corresponding to the structure, and register the tissue type model with the structure, wherein each relaxation model corresponds to a tissue type.

Example 31-A method of detecting temperature in an object of interest comprised of a first tissue type with a magnetic resonance imaging apparatus. The method comprising obtaining calibration data comprising T2 data for different temperatures for different tissue types including the first tissue type, generating models for the different tissue types from the calibration data, positioning the object of interest in a field of view, and transmitting a pulse sequence comprising sweeping frequency pulses. The method further comprises receiving T2 data corresponding to the object of interest, generating a T2-weighted image of the object of interest, and converting the T2-weighed image of the object of interest to a heat map based on the model for the first tissue type.

Example 32-The method of Example 31, further comprising receiving a first input identifying the first tissue type of the object of interest.

Example 33-The method of Examples 31 or 32, further comprising an RF coil set transmitting the pulse sequence comprising sweeping frequency pulses and receiving T2 data corresponding to the object of interest.

Example 34-The method of Examples 31, 32, or 33, wherein the magnetic resonance imaging apparatus comprises a permanent magnet assembly, and further comprising the permanent magnet assembly generating an inherent gradient magnetic field into the field of view.

While several forms have been illustrated and described, it is not the intention of Applicant to restrict or limit the scope of the appended claims to such detail. Numerous modifications, variations, changes, substitutions, combinations, and equivalents to those forms may be implemented and will occur to those skilled in the art without departing from the scope of the present disclosure. Moreover, the structure of each element associated with the described forms can be alternatively described as a means for providing the function performed by the element. Also, where materials are disclosed for certain components, other materials may be used. It is therefore to be understood that the foregoing description and the appended claims are intended to cover all such modifications, combinations, and variations as falling within the scope of the disclosed forms. The appended claims are intended to cover all such modifications, variations, changes, substitutions, modifications, and equivalents.

The foregoing detailed description has set forth various forms of the devices and/or processes via the use of block diagrams, flowcharts, and/or examples. Insofar as such block diagrams, flowcharts, and/or examples contain one or more functions and/or operations, it will be understood by those within the art that each function and/or operation within such block diagrams, flowcharts, and/or examples can be implemented, individually and/or collectively, by a wide range of hardware, software, firmware, or virtually any combination thereof. Those skilled in the art will recognize that some aspects of the forms disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and or firmware would be well within the skill of one of skill in the art in light of this disclosure. In addition, those skilled in the art will appreciate that the mechanisms of the subject matter described herein are capable of being distributed as one or more program products in a variety of forms, and that an illustrative form of the subject matter described herein applies regardless of the particular type of signal bearing medium used to actually carry out the distribution.

Instructions used to program logic to perform various disclosed aspects can be stored within a memory in the system, such as dynamic random access memory (DRAM), cache, flash memory, or other storage. Furthermore, the instructions can be distributed via a network or by way of other computer readable media. Thus a machine-readable medium may include any mechanism for storing or transmitting information in a form readable by a machine (e.g., a computer), but is not limited to, floppy diskettes, optical disks, compact disc, read-only memory (CD-ROMs), and magneto-optical disks, read-only memory (ROMs), random access memory (RAM), erasable programmable read-only memory (EPROM), electrically erasable programmable read-only memory (EEPROM), magnetic or optical cards, flash memory, or a tangible, machine-readable storage used in the transmission of information over the Internet via electrical, optical, acoustical or other forms of propagated signals (e.g., carrier waves, infrared signals, digital signals, etc.). Accordingly, the non-transitory computer-readable medium includes any type of tangible machine-readable medium suitable for storing or transmitting electronic instructions or information in a form readable by a machine (e.g., a computer).

As used in any aspect herein, the term “control circuit” may refer to, for example, hardwired circuitry, programmable circuitry (e.g., a computer processor including one or more individual instruction processing cores, processing unit, processor, microcontroller, microcontroller unit, controller, digital signal processor (DSP), programmable logic device (PLD), programmable logic array (PLA), or field programmable gate array (FPGA)), state machine circuitry, firmware that stores instructions executed by programmable circuitry, and any combination thereof. The control circuit may, collectively or individually, be embodied as circuitry that forms part of a larger system, for example, an integrated circuit (IC), an application-specific integrated circuit (ASIC), a system on-chip (SoC), desktop computers, laptop computers, tablet computers, servers, smart phones, etc. Accordingly, as used herein “control circuit” includes, but is not limited to, electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e.g., forms of random access memory), and/or electrical circuitry forming a communications device (e.g., a modem, communications switch, or optical-electrical equipment). Those having skill in the art will recognize that the subject matter described herein may be implemented in an analog or digital fashion or some combination thereof.

As used in any aspect herein, the term “logic” may refer to an app, software, firmware and/or circuitry configured to perform any of the aforementioned operations. Software may be embodied as a software package, code, instructions, instruction sets and/or data recorded on non-transitory computer readable storage medium. Firmware may be embodied as code, instructions or instruction sets and/or data that are hard-coded (e.g., nonvolatile) in memory devices.

As used in any aspect herein, the terms “component,” “system,” “module” and the like can refer to a computer-related entity, either hardware, a combination of hardware and software, software, or software in execution.

As used in any aspect herein, an “algorithm” refers to a self-consistent sequence of steps leading to a desired result, where a “step” refers to a manipulation of physical quantities and/or logic states which may, though need not necessarily, take the form of electrical or magnetic signals capable of being stored, transferred, combined, compared, and otherwise manipulated. It is common usage to refer to these signals as bits, values, elements, symbols, characters, terms, numbers, or the like. These and similar terms may be associated with the appropriate physical quantities and are merely convenient labels applied to these quantities and/or states.

A network may include a packet switched network. The communication devices may be capable of communicating with each other using a selected packet switched network communications protocol. One example communications protocol may include an Ethernet communications protocol which may be capable permitting communication using a Transmission Control Protocol/Internet Protocol (TCP/IP). The Ethernet protocol may comply or be compatible with the Ethernet standard published by the Institute of Electrical and Electronics Engineers (IEEE) titled “IEEE 802.3 Standard”, published in December 2008 and/or later versions of this standard. Alternatively or additionally, the communication devices may be capable of communicating with each other using an X.25 communications protocol. The X.25 communications protocol may comply or be compatible with a standard promulgated by the International Telecommunication Union-Telecommunication Standardization Sector (ITU-T). Alternatively or additionally, the communication devices may be capable of communicating with each other using a frame relay communications protocol. The frame relay communications protocol may comply or be compatible with a standard promulgated by Consultative Committee for International Telegraph and Telephone (CCITT) and/or the American National Standards Institute (ANSI). Alternatively or additionally, the transceivers may be capable of communicating with each other using an Asynchronous Transfer Mode (ATM) communications protocol. The ATM communications protocol may comply or be compatible with an ATM standard published by the ATM Forum titled “ATM-MPLS Network Interworking 2.0” published August 2001, and/or later versions of this standard. Of course, different and/or after-developed connection-oriented network communication protocols are equally contemplated herein.

Unless specifically stated otherwise as apparent from the foregoing disclosure, it is appreciated that, throughout the foregoing disclosure, discussions using terms such as “processing,” “computing,” “calculating,” “determining,” “displaying,” or the like, refer to the action and processes of a computer system, or similar electronic computing device, that manipulates and transforms data represented as physical (electronic) quantities within the computer system's registers and memories into other data similarly represented as physical quantities within the computer system memories or registers or other such information storage, transmission or display devices.

One or more components may be referred to herein as “configured to,” “configurable to,” “operable/operative to,” “adapted/adaptable,” “able to,” “conformable/conformed to,” etc. Those skilled in the art will recognize that “configured to” can generally encompass active-state components and/or inactive-state components and/or standby-state components, unless context requires otherwise.

The terms “proximal” and “distal” are used herein with reference to a magnetic imaging apparatus. The term “proximal” refers to a direction toward the magnetic imaging apparatus and “distal” refers to a direction away from the magnetic imaging apparatus. It will be further appreciated that, for convenience and clarity, spatial terms such as “vertical”, “horizontal”, “up”, and “down” may be used herein with respect to the drawings. However, these terms are not intended to be limiting and/or absolute.

Those skilled in the art will recognize that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to claims containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations.

In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that typically a disjunctive word and/or phrase presenting two or more alternative terms, whether in the description, claims, or drawings, should be understood to contemplate the possibilities of including one of the terms, either of the terms, or both terms unless context dictates otherwise. For example, the phrase “A or B” will be typically understood to include the possibilities of “A” or “B” or “A and B.”

With respect to the appended claims, those skilled in the art will appreciate that recited operations therein may generally be performed in any order. Also, although various operational flow diagrams are presented in a sequence(s), it should be understood that the various operations may be performed in other orders than those which are illustrated, or may be performed concurrently. Examples of such alternate orderings may include overlapping, interleaved, interrupted, reordered, incremental, preparatory, supplemental, simultaneous, reverse, or other variant orderings, unless context dictates otherwise. Furthermore, terms like “responsive to,” “related to,” or other past-tense adjectives are generally not intended to exclude such variants, unless context dictates otherwise.

It is worthy to note that any reference to “one aspect,” “an aspect,” “an exemplification,” “one exemplification,” and the like means that a particular feature, structure, or characteristic described in connection with the aspect is included in at least one aspect. Thus, appearances of the phrases “in one aspect,” “in an aspect,” “in an exemplification,” and “in one exemplification” in various places throughout the specification are not necessarily all referring to the same aspect. Furthermore, the particular features, structures or characteristics may be combined in any suitable manner in one or more aspects.

Any patent application, patent, non-patent publication, or other disclosure material referred to in this specification and/or listed in any Application Data Sheet is incorporated by reference herein, to the extent that the incorporated materials is not inconsistent herewith. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

In summary, numerous benefits have been described which result from employing the concepts described herein. The foregoing description of the one or more forms has been presented for purposes of illustration and description. It is not intended to be exhaustive or limiting to the precise form disclosed. Modifications or variations are possible in light of the above teachings. The one or more forms were chosen and described in order to illustrate principles and practical application to thereby enable one of ordinary skill in the art to utilize the various forms and with various modifications as are suited to the particular use contemplated. It is intended that the claims submitted herewith define the overall scope.

What is claimed is:

1. A magnetic imaging apparatus, comprising:
a housing comprising a face, wherein a first axis extends through the face into a field of view;
an array of permanent magnets positioned in the housing, wherein an inherent gradient magnetic field extends from the array of permanent magnets relative to the first axis into the field of view;
a gradient coil set;
at least one radio frequency coil;
a power circuit;
a memory storing a relaxation model for a tissue type; and
a control circuit in signal communication with the gradient coil set, the at least one radio frequency coil, the power circuit, and the memory, wherein the control circuit is configured to:
obtain a T2 data set related to a structure positioned in the field of view, wherein the structure corresponds to the tissue type;
generate a T2-weighted image of the structure; and
convert the T2-weighed image of the structure to a heat map based on the relaxation model for the tissue type.

2. The magnetic imaging apparatus of claim 1, wherein the relaxation model comprises a monoexponential model.

3. The magnetic imaging apparatus of claim 1, wherein the gradient coil and the at least one radio frequency coil are coupled to the power circuit.

4. The magnetic imaging apparatus of claim 1, wherein the control circuit is further configured to transmit a pulse sequence comprising a plurality of sweeping frequency pulses.

5. The magnetic imaging apparatus of claim 4, wherein the T2 data set is generated from the plurality of sweeping frequency pulses.

6. The magnetic imaging apparatus of claim 4, wherein each sweeping frequency pulse produces an echo comprising a duration between 2 milliseconds and 20 milliseconds.

7. The magnetic imaging apparatus of claim 6, wherein the plurality of sweeping frequency pulses produces between 10 echoes and 100 echoes.

8. The magnetic imaging apparatus of claim 6, wherein each sweeping frequency pulse comprises a bandwidth between 10 KHz and 200 KHz.

9. The magnetic imaging apparatus of claim 1, wherein the radio frequency coil is configured to transmit pulses having a frequency between 1 Megahertz and 21 Megahertz.

10. The magnetic imaging apparatus of claim 1, wherein the magnetic field strength in the field of view is less than 1 Tesla.

11. The magnetic imaging apparatus of claim 1, wherein the inhomogeneity of the magnetic field in the field of view is between 200 ppm and 200,000 ppm.

12. The magnetic imaging apparatus of claim 1, wherein the relaxation model is generated from calibration data comprising a plurality of T2 relaxation data for different temperatures for the tissue type.

13. The magnetic imaging apparatus of claim 1, wherein the magnetic imaging apparatus is a single-sided magnetic imaging apparatus, and wherein the housing and the gradient coil set are positioned on a first side of field of view.

14. The magnetic imaging apparatus of claim 1, further comprising a user input device configured to receive the tissue type of the structure.

15. The magnetic imaging apparatus of claim 1, further comprising an automated tissue type recognition module configured to:
identify a tissue type model corresponding to the structure; and
register the tissue type model with the structure.

16. A single-sided magnetic imaging apparatus, comprising:
a housing comprising an array of permanent magnets, wherein an inherent gradient magnetic field extends from the array of permanent magnets relative to a first axis into a field of view, wherein the field of view is adjacent to the housing;
a radio frequency coil;
a power circuit coupled to the radio frequency coil;
a memory storing relaxation models for tissue at different temperatures; and
a control circuit in signal communication with the radio frequency coil, the power circuit, and the memory, wherein the control circuit is configured to:
transmit a waveform sequence to the radio frequency coil to produce an echo train sequence;
obtain a T2 data set related to a structure positioned in the field of view;
generate a T2-weighted image of the structure; and
convert the T2-weighed image of the structure to a heat map based on the relaxation models stored in the memory.

17. The single-sided magnetic imaging apparatus of claim 16, wherein the relaxation models comprise a monoexponential model.

18. The single-sided magnetic imaging apparatus of claim 16, wherein the echo train sequence comprises phase encodes.

19. The single-sided magnetic imaging apparatus of claim 16, wherein the T2 data set is generated based on the echo train sequence.

20. The single-sided magnetic imaging apparatus of claim 16, wherein the echo train sequence comprises echoes comprising a duration between 2 milliseconds and 20 milliseconds.

21. The single-sided magnetic imaging apparatus of claim 16, wherein the echo train sequence comprises between 10 echoes and 100 echoes.

22. The single-sided magnetic imaging apparatus of claim 16, wherein the echo train sequence is produced by a plurality of sweeping frequency pulses comprising a bandwidth between 10 KHz and 200 KHz.

23. The single-sided magnetic imaging apparatus of claim 16, wherein the radio frequency coil is configured to transmit pulses having a frequency between 1 Megahertz and 21 Megahertz.

24. The single-sided magnetic imaging apparatus of claim 16, wherein the magnetic field strength in the field of view is less than 1 Tesla.

25. The single-sided magnetic imaging apparatus of claim 16, wherein the inhomogeneity of the magnetic field in the field of view is between 200 ppm and 200,000 ppm.

26. The single-sided magnetic imaging apparatus of claim 16, wherein the relaxation models are generated from calibration data comprising a plurality of T2 data at different temperatures for different tissue types.

27. The single-sided magnetic imaging apparatus of claim 16, further comprising a gradient coil set positioned in the housing, wherein the gradient coil set, the radio frequency coil, and the housing are positioned on a first side of the field of view.

28. The single-sided magnetic imaging apparatus of claim 16, wherein the radio frequency coil comprises a radio frequency transmission coil, and wherein the single-sided magnetic imaging apparatus further comprises a radio frequency reception coil.

29. The single-sided magnetic imaging apparatus of claim 16, further comprising a user input device configured to receive the tissue type of the structure, wherein each relaxation model corresponds to a tissue type.

30. The single-sided magnetic imaging apparatus of claim 16, further comprising an automated tissue type recognition module configured to:
identify a tissue type model corresponding to the structure; and
register the tissue type model with the structure, wherein each relaxation model corresponds to a tissue type.

31. A method of detecting temperature in an object of interest comprised of a first tissue type with a magnetic resonance imaging apparatus, the method comprising:
obtaining calibration data comprising T2 data for different temperatures for different tissue types including the first tissue type;
generating models for the different tissue types from the calibration data;
positioning the object of interest in a field of view;
transmitting a pulse sequence comprising sweeping frequency pulses;
receiving T2 data corresponding to the object of interest;
generating a T2-weighted image of the object of interest; and
converting the T2-weighed image of the object of interest to a heat map based on the model for the first tissue type.

32. The method of claim 31, further comprising receiving a first input identifying the first tissue type of the object of interest.

33. The method of claim 31, further comprising an RF coil set transmitting the pulse sequence comprising sweeping frequency pulses and receiving T2 data corresponding to the object of interest.

34. The method of claim 31, wherein the magnetic resonance imaging apparatus comprises a permanent magnet assembly, and further comprising the permanent magnet assembly generating an inherent gradient magnetic field into the field of view.
